SEQUENCE LISTING (37 sequences)

SEQ ID NO: 1 - protein, 72 aa, Finegoldia magna
PKEEVTIKANLIYADGKTQTAEFKGTFEEAAAEAYRYADALKKDNGEYTVDVADKGYTLNIKFAGKEKTPEE

SEQ ID NO: 2 - protein, 72 aa, synthetic construct
  REGION 1..72: note = Recombinantly produced
PKEEVTIKAHLIYADGKTQTAEFKGTFEEAAAEAYRYADALKKDHGEYTVDVADKGYTLYIKFAGKEKTPEE

SEQ ID NO: 3 - protein, 72 aa, synthetic construct
  REGION 1..72: note = Recombinantly produced
PKEEVTIKAHLIYADGKTQTAEFKGTFEEAAAEAYRYADALKKDHGEYTVDVADKGYTLQIKFAGKEKTPEE

SEQ ID NO: 4 - protein, 72 aa, synthetic construct
  REGION 1..72: note = Recombinantly produced
PKEEVTIKAHLIYADGKTQTAEFKGTFEEATAEAYRYADLLAKEHGKYTVDVADKGYTLYIKFAGKEKTPEE

SEQ ID NO: 5 - protein, 72 aa, synthetic construct
  REGION 1..72: note = Recombinantly produced
PKEEVTIKAHLIYADGKTQTAEFKGTFEEATAEAYRYADLLAKEHGKYTVDVADKGYTLQIKFAGKEKTPEE

SEQ ID NO: 6 - protein, 72 aa, synthetic construct
  REGION 1..72: note = Recombinantly produced
SEEEVTIKAHLIFANGSTQTAEFKGTFEKATSEAYAYADTLKKDHGEYTVDVADKGYTLYIKFAGKEKTPEE

SEQ ID NO: 7 - protein, 72 aa, synthetic construct
  REGION 1..72: note = Recombinantly produced
SEEEVTIKAHLIFANGSTQTAEFKGTFEKATSEAYAYADTLKKDHGEYTVDVADKGYTLQIKFAGKEKTPEE

SEQ ID NO: 8 - protein, 74 aa, synthetic construct
  REGION 1..74: note = Recombinantly produced
PKEEVTIKAHLIYADGKTQTAEFKGTFAEATAEAYRYADLLAKEHGKYTADLEDGGYTIYIRFAGKKVDEKPEE

SEQ ID NO: 9 - protein, 74 aa, synthetic construct
  REGION 1..74: note = Recombinantly produced
PKEEVTIKAHLIYADGKTQTAEFKGTFAEATAEAYRYADLLAKEHGKYTADLEDGGYTIQIRFAGKKVDEKPEE

SEQ ID NO: 10 - protein, 70 aa, synthetic construct
  REGION 1..70: note = Recombinantly produced
KEQVTIKEHIYFEDGTVQTATFKGTFAEATAEAYRYADLLSKEHGKYTADLEDGGYTIQIRFAGKEEPEE

SEQ ID NO: 11 - protein, 70 aa, synthetic construct; identical to SEQ ID NO: 10
  REGION 1..70: note = Recombinantly produced

SEQ ID NO: 12 - protein, 74 aa, synthetic construct
  REGION 1..74: note = Recombinantly produced
PKEEVTIKVHLIFADGKTQTAEFKGTFEEATAKAYAYADLLAKEHGEYTADLEDGGYTIYIKFAGKETPETPEE

SEQ ID NO: 13 - protein, 74 aa, synthetic construct
  REGION 1..74: note = Recombinantly produced
PKEEVTIKVHLIFADGKTQTAEFKGTFEEATAKAYAYADLLAKEHGEYTADLEDGGYTIQIKFAGKETPETPEE

SEQ ID NO: 14 - protein, 74 aa, synthetic construct
  REGION 1..74: note = Recombinantly produced
PKEEVTIKVHLIFADGKIQTAEFKGTFEEATAKAYAYADLLAKEHGEYTADLEDGGYTIYIKFAGKETPETPEE

SEQ ID NO: 15 - protein, 74 aa, synthetic construct
  REGION 1..74: note = Recombinantly produced
PKEEVTIKVHLIFADGKIQTAEFKGTFEEATAKAYAYADLLAKEHGEYTADLEDGGYTIQIKFAGKETPETPEE

SEQ ID NO: 16 - protein, 74 aa, synthetic construct
  REGION 1..74: note = Recombinantly produced
PKEEVTIKVHLIFADGKTQTAEFKGTFEEATAEAYRYADLLAKVHGEYTADLEDGGYTIYIKFAGKEQPGENPG

SEQ ID NO: 17 - protein, 74 aa, synthetic construct
  REGION 1..74: note = Recombinantly produced
PKEEVTIKVHLIFADGKTQTAEFKGTFEEATAEAYRYADLLAKVHGEYTADLEDGGYTIQIKFAGKEQPGENPG

SEQ ID NO: 18 - protein, 72 aa, synthetic construct
  REGION 1..72: note = Recombinantly produced
PKEEVTIKAHLIFADGKTQTAEFKGTFEEATAEAYRYADLLAKVHGEYTADLEDGGYTIYIKFAGKEQPGEN

SEQ ID NO: 19 - protein, 72 aa, synthetic construct
  REGION 1..72: note = Recombinantly produced
PKEEVTIKAHLIFADGKTQTAEFKGTFEEATAEAYRYADLLAKVHGEYTADLEDGGYTIQIKFAGKEQPGEN

SEQ ID NO: 20 - protein, 61 aa, synthetic construct
  REGION 1..61: note = Recombinantly produced
VTIKAHLIFANGSTQTAEFKGTFEKATSEAYAYADTLKKDHGEYTVDVADKGYTLYIKFAG

SEQ ID NO: 21 - protein, 61 aa, synthetic construct
  REGION 1..61: note = Recombinantly produced
VTIKAHLIFANGSTQTAEFKGTFEKATSEAYAYADTLKKDHGEYTVDVADKGYTLQIKFAG

SEQ ID NO: 22 - protein, 61 aa, synthetic construct
  REGION 1..61: note = Recombinantly produced
VTIKAHLIYADGKTQTAEFKGTFEEAAAEAYRYADALKKDHGEYTVDVADKGYTLYIKFAG

SEQ ID NO: 23 - protein, 61 aa, synthetic construct
  REGION 1..61: note = Recombinantly produced
VTIKAHLIYADGKTQTAEFKGTFEEAAAEAYRYADALKKDHGEYTVDVADKGYTLQIKFAG

SEQ ID NO: 24 - protein, 61 aa, synthetic construct
  REGION 1..61: note = Recombinantly produced
VTIKAHLIYADGKTQTAEFKGTFEEATAEAYRYADLLAKEHGKYTVDVADKGYTLYIKFAG

SEQ ID NO: 25 - protein, 61 aa, synthetic construct
  REGION 1..61: note = Recombinantly produced
VTIKAHLIYADGKTQTAEFKGTFEEATAEAYRYADLLAKEHGKYTVDVADKGYTLQIKFAG

SEQ ID NO: 26 - protein, 61 aa, synthetic construct
  REGION 1..61: note = Recombinantly produced
VTIKAHLIYADGKTQTAEFKGTFAEATAEAYRYADLLAKEHGKYTADLEDGGYTIYIRFAG

SEQ ID NO: 27 - protein, 61 aa, synthetic construct
  REGION 1..61: note = Recombinantly produced
VTIKAHLIYADGKTQTAEFKGTFAEATAEAYRYADLLAKEHGKYTADLEDGGYTIQIRFAG

SEQ ID NO: 28 - protein, 61 aa, synthetic construct
  REGION 1..61: note = Recombinantly produced
VTIKEHIYFEDGTVQTATFKGTFAEATAEAYRYADLLSKEHGKYTADLEDGGYTIYIRFAG

SEQ ID NO: 29 - protein, 61 aa, synthetic construct
  REGION 1..61: note = Recombinantly produced
VTIKEHIYFEDGTVQTATFKGTFAEATAEAYRYADLLSKEHGKYTADLEDGGYTIQIRFAG

SEQ ID NO: 30 - protein, 61 aa, synthetic construct
  REGION 1..61: note = Recombinantly produced
VTIKVHLIFADGKTQTAEFKGTFEEATAKAYAYADLLAKEHGEYTADLEDGGYTIYIKFAG

SEQ ID NO: 31 - protein, 61 aa, synthetic construct
  REGION 1..61: note = Recombinantly produced
VTIKVHLIFADGKTQTAEFKGTFEEATAKAYAYADLLAKEHGEYTADLEDGGYTIQIKFAG

SEQ ID NO: 32 - protein, 61 aa, synthetic construct
  REGION 1..61: note = Recombinantly produced
VTIKVHLIFADGKIQTAEFKGTFEEATAKAYAYADLLAKEHGEYTADLEDGGYTIYIKFAG

SEQ ID NO: 33 - protein, 61 aa, synthetic construct
  REGION 1..61: note = Recombinantly produced
VTIKVHLIFADGKIQTAEFKGTFEEATAKAYAYADLLAKEHGEYTADLEDGGYTIQIKFAG

SEQ ID NO: 34 - protein, 61 aa, synthetic construct
  REGION 1..61: note = Recombinantly produced
VTIKVHLIFADGKTQTAEFKGTFEEATAEAYRYADLLAKVHGEYTADLEDGGYTIYIKFAG

SEQ ID NO: 35 - protein, 61 aa, synthetic construct
  REGION 1..61: note = Recombinantly produced
VTIKVHLIFADGKTQTAEFKGTFEEATAEAYRYADLLAKVHGEYTADLEDGGYTIQIKFAG

SEQ ID NO: 36 - protein, 61 aa, synthetic construct
  REGION 1..61: note = Recombinantly produced
VTIKAHLIFADGKTQTAEFKGTFEEATAEAYRYADLLAKVHGEYTADLEDGGYTIYIKFAG

SEQ ID NO: 37 - protein, 61 aa, synthetic construct
  REGION 1..61: note = Recombinantly produced
VTIKAHLIFADGKTQTAEFKGTFEEATAEAYRYADLLAKVHGEYTADLEDGGYTIQIKFAG

(19) United States
(12) Patent Application Publication
(10) Pub. No.: US 2026/0258092 A1
(43) Pub. Date: Sep. 3, 2026

(54) KAPPA LIGHT CHAIN-BINDING CONVECTION MATRIX

(71) Applicant: Cytiva BioProcess R&D AB, Uppsala (SE)

(72) Inventors: Ronnie Palmgren, Uppsala (SE); Keyhan Esfandiarfard, Uppsala (SE); Tania Ahmad, Uppsala (SE); Tomas Björkman, Uppsala (SE); Lisa Marx, Uppsala (SE)

(21) Appl. No.: 18/876,100

(22) PCT Filed: Jun. 20, 2023
(86) PCT No.: PCT/EP2023/066527

§ 371 Date: Dec. 17, 2024

(30) Foreign Application Priority Data
Jun. 22, 2022 (SE) 2250770-1

(51) Int. Cl. C07K 14/195; B01D 15/38; B01J 20/28; B01J 20/285; B01J 20/289; B01J 20/32; C07K 1/22; C07K 16/32; C07K 17/12

(52) U.S. Cl. CPC C07K 14/195; B01D 15/3809; B01J 20/28023; B01J 20/285; B01J 20/289; B01J 20/3208; B01J 20/3219; B01J 20/3274; C07K 1/22; C07K 16/32; C07K 17/12

(57) ABSTRACT

The present invention relates to isolation and separation of kappa light chain-containing proteins. More specifically it relates to a separation matrix comprising kappa light chain-binding ligands covalently coupled to a porous support, wherein said kappa light chain-binding ligands comprise, consists essentially of, or consists of multimers of alkali-stabilized Finegoldia magna (formerly Peptostreptococcus Magnus) Protein L domains; and said porous support is a convection-based chromatography matrix.

### TECHNICAL FIELD OF THE INVENTION

The present invention relates to the field of separation of biomolecules. More specifically, it relates to a separation matrix for affinity chromatography and separation of biomolecules based on the presence of a kappa light chain, such as immunoglobulins and immunoglobulin fractions. The invention also relates to methods of using said separation matrix.

### BACKGROUND

Immunoglobulins and immunoglobulin fragments represent the most prevalent biopharmaceutical products in either manufacture or development worldwide. The high commercial demand for, and hence value of, this particular therapeutic market has led to the emphasis being placed on pharmaceutical companies to maximize the productivity of their respective manufacturing processes whilst controlling the associated costs.

Affinity chromatography, typically on matrices comprising staphylococcal Protein A or variants thereof, is normally used as one of the key steps in the purification of intact immunoglobulin molecules. The highly selective binding of Protein A to the Fc chain of immunoglobulins provides for a generic step with very high clearance of impurities and contaminants.

For immunoglobulin fragments, or antibody fragments, such as Fab, single-chain variable fragments (scFv), bi-specific T-cell engagers (BiTEs), domain antibodies etc., which lack the Fc chain but have a subclass 1, 3 or 4 kappa light chain, matrices comprising Protein L derived from Finegoldia magna (formerly Peptostreptococcus Magnus) (B Åkerström, L Björck: J. Biol. Chem. 264, 19740-19746, 1989; W Kastem et al: J. Biol. Chem. 267, 12820-12825, 1992; B H K Nilson et al: J. Biol. Chem. 267, 2234-2239, 1992 and U.S. Pat. No. 6,822,075) show great promise as a purification platform providing the high selectivity needed.

Protein L matrices are commercially available as for instance Capto™ L from Cytiva™ and can be used for separation of kappa light chain-containing proteins such as intact antibodies, Fab fragments, scFv fragments, domain antibodies etc. About 75% of the antibodies produced by healthy humans have a kappa light chain and about 90% of therapeutic monoclonal antibodies and antibody fragments contain kappa light chains (Carter, P., Lazar, G. Next generation antibody drugs: pursuit of the ‘high-hanging fruit’. Nat Rev Drug Discov 17, 197-223 (2018). https://doi.org/10.1038/nrd.2017.227).

Any bioprocess chromatography application requires comprehensive attention to definite removal of impurities and/or contaminants. Such impurities and/or contaminants can for example be non-eluted molecules adsorbed to the stationary phase or matrix in a chromatographic procedure, such as non-desired biomolecules or microorganisms, including for example proteins, carbohydrates, lipids, bacteria and viruses. The removal of such impurities and/or contaminants from the matrix is usually performed after a first elution of the desired product in order to regenerate the matrix before subsequent use. Such removal usually involves a procedure known as cleaning-in-place (CIP), wherein agents capable of either inactivating or eluting impurities from the stationary phase are used. One such class of agents often used with chromatography media is alkaline solutions that are passed over the matrix. At present the most extensively used cleaning and sanitizing agent is NaOH, and it is desirable to use it in concentrations ranging from 0.05 up to e.g. 1 M, depending on the degree and nature of contamination and impurity. Protein L is however a rather alkali-sensitive protein compared to e.g. Protein A and only tolerates up to about 15 mM NaOH over a large number of cycles. This means that additional, less desirable cleaning solutions, e.g. urea or guanidinium salts, may also have to be used in order to ensure sufficient cleaning.

There is thus still a need in this field to obtain a separation matrix containing Protein L-derived ligands having an improved stability towards alkaline cleaning procedures.

### SUMMARY

The present inventors have attained to solve the above-mentioned problem by providing a separation matrix comprising kappa light chain-binding ligands covalently coupled to a porous support, wherein said kappa light chain-binding ligands comprise, consists essentially of, or consists of multimers of alkali-stabilized Finegoldia magna (formerly Peptostreptococcus Magnus) Protein L domains; and said porous support is a convection-based chromatography matrix.

The convection-based chromatography matrix may be a fibrous substrate. Said fibrous substrate may be based on electrospun polymeric fibers or cellulose fibers, optionally non-woven fibers.

The polymer may be selected from the group consisting of cellulose, cellulose acetate, polysulfones, polyamides, polyacrylic acid, polymethacrylic acid, polyacrylonitrile, polystyrene, polyethylene oxide, and mixtures thereof. According to one embodiment, the fibrous substrate is a fibrous non-woven polymer matrix.

The fibers comprised in said fibrous substrate may have a cross-sectional diameter of 10-1000 nm, such as 200-800 nm, 200-400 nm or 300-400 nm.

The ligands may be bound to divinyl sulfone functional groups coupled to glycidol groups grafted onto the fibrous substrate.

Said kappa light chain-binding ligands may comprise at least two alkali-stabilized Protein L domains. The alkali-stabilized Protein L domains may be selected from the group comprising of functional variants of a B1 domain, a B2 domain, a B3 domain, a B4 domain, a B5 domain, a C2 domain, a C3 domain, a C4 domain and a D1 domain of Finegoldia magna (formerly Peptostreptococcus Magnus) Protein L, wherein the positions which in an alignment corresponds to positions 10 and 45 in a B2 domain (SEQ ID NO 1) are histidine, and the position which in an alignment corresponds to position 60 in a B2 domain (SEQ ID NO 1) is a tyrosine or a glutamine. In one embodiment, the alkali-stabilized Protein L domains are chosen from the group comprising a B2 domain, a B3 domain, a B4 domain, a C2 domain, a C3 domain, a C4 domain and a D1 domain.

The alkali-stabilized Protein L domains may have at least 90%, 95% or 98% sequence identity or a 77.5% sequence similarity as determined by BLOSUM matrix of 75, with a gap open penalty of 12, a gap extension penalty of 3, with any one of the amino acid sequences SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18 or SEQ ID NO 19, wherein the positions which in an alignment corresponds to positions 10 and 45 in SEQ ID NO 1, and the position which in an alignment corresponds to position 60 in SEQ ID NO 1 are not variable. The alkali-stabilized Protein L domains may have at least 90%, 95% or 98% sequence identity, or a 77.5% sequence similarity as determined by BLOSUM matrix of 75, with a gap open penalty of 12, a gap extension penalty of 3, with any one of the amino acid sequences SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36 or SEQ ID NO:37.

The ligand density for the separation matrix may be at least 20 mg/ml porous support, or at least 25 mg/ml porous support, or at least 30 mg/ml porous support, or at least 35 mg/ml porous support, or at least 40 mg/ml porous support, or at least 45 mg/ml porous support, or at least 50 mg/ml porous support.

The separation matrix may have a dynamic binding capacity (DBC) of a kappa light chain-comprising antibody, such as Trastuzumab, of 25 g/mL at 10% breakthrough when running at a flow of 10 mL/min in a 0.4 mL HiTrap™ device.

The separation matrix may have a dynamic binding capacity (DBC) of a kappa light chain-comprising antibody, such as Trastuzumab, of 45 g/mL at 10% breakthrough when running at a flow of 10 mL/min in a 0.4 mL HiTrap™ device.

Additionally, there is provided a method of isolating a kappa light chain-containing protein comprising the steps of: a) contacting a liquid sample comprising a kappa light chain-containing protein with a separation matrix; b) washing said separation matrix with one or a combination of several washing liquids; c) eluting the kappa light chain-containing protein from the separation matrix with an elution liquid; and d) cleaning the separation matrix with a cleaning liquid; wherein the separation matrix has a dynamic binding capacity (DBC) of a kappa light chain-comprising antibody, such as Trastuzumab, of 25 g/mL at 10% breakthrough when running at a flow of 10 mL/min in a 0.4 mL HiTrap™ device.

Furthermore, there is provided a method for separation of isolating a kappa light chain-binding protein from lambda light chain-containing proteins, comprising the steps of: a) contacting a liquid sample comprising a kappa light chain-containing protein with a separation matrix; b) washing said separation matrix with one or a combination of several washing liquids; c) eluting the kappa light chain-containing protein from the separation matrix with an elution liquid; and d) cleaning the separation matrix with a cleaning liquid; wherein the separation matrix has a dynamic binding capacity (DBC) of a kappa light chain-comprising antibody, such as Trastuzumab, of 25 g/mL at 10% breakthrough when running at a flow of 10 mL/min in a 0.4 mL HiTrap™ device.

Additionally, there is provided a method for separation of bispecific antibodies from mono-specific antibodies, comprising the steps of: a) contacting a liquid sample comprising a bispecific antibody with a separation matrix; b) washing said separation matrix with one or a combination of several washing liquids; c) eluting the bispecific antibody from the separation matrix with an elution liquid; and d) cleaning the separation matrix with a cleaning liquid; wherein the separation matrix has a dynamic binding capacity (DBC) of a kappa light chain-comprising antibody, such as Trastuzumab, of 25 g/mL at 10% breakthrough when running at a flow of 10 mL/min in a 0.4 mL HiTrap™ device.

In any of the separation methods disclosed above, the separation is performed by applying a volume gradient or a pH gradient in step c).

In any of the methods according to the above, the separation matrix may be according to the above.

### DRAWINGS

FIG. 1 schematically depicts the HiTrap™ devices (Cytiva™) used for the DBC comparison between prototypes and a commercial product in Example 1.

FIG. 2 shows a chromatogram for Fab and Trastuzumab for a pH gradient slope of 20 mL

FIG. 3 shows a chromatogram for Fab and Trastuzumab for a pH gradient slope of 40 mL

FIG. 4 shows a chromatogram for Fab and Trastuzumab for a pH gradient slope of 60 mL

FIG. 5 shows a chromatogram for Fab and Trastuzumab for a pH gradient slope of 88 mL

### DEFINITIONS

The terms “antibody” and “immunoglobulin” may be used interchangeably herein and refers to an antigen-binding protein having a basic four-polypeptide chain structure consisting of two heavy (H) chains and two light (L) chains, said chains being stabilized by interchain or intrachain disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (VL) and a light chain constant region. The light chain constant region is comprised of one domain, CL. There are two types of light chain in humans, kappa chain and lambda chain. The term is to be understood to include any antibody, including but not limited to monoclonal antibodies and bi-specific antibodies, as well as fragments of antibodies, fusion proteins comprising antibodies or antibody fragments and conjugates comprising antibodies or antibody fragments.

The terms a “kappa light chain-binding polypeptide” and “kappa light chain-binding protein” herein mean a polypeptide or protein respectively, capable of binding to a subclass 1, 3 or 4 kappa light chain of an antibody (also called V_{kI}, V_{kIII} and V_{kIV}, as in B H K Nilson et al: J. Biol. Chem. 267, 2234-2239, 1992), and include e.g. Protein L, and any variant, fragment or fusion protein thereof that has maintained said binding property.

The term “kappa light chain-containing protein” is used as a synonym of “immunoglobulin kappa light chain-containing protein” and herein means a protein comprising a subclass 1, 3 or 4 kappa light chain (also called V_{kI}, V_{kIII} and V_{kIV}, as in B H K Nilson et al: J. Biol. Chem. 267, 10 2234-2239, 1992) derived from an antibody and includes any intact antibodies, antibody fragments, fusion proteins, conjugates or recombinant proteins containing a subclass 1, 3 or 4 kappa light chain.

The term “mAb” stands for monoclonal antibody

The term “Fab” stands for an antigen binding fragment from an immunoglobulin, comprising a kappa light chain or a lambda light chain.

The term “bi-specific antibody” stands for an antibody that can bind to two different types of antigen or two different epitopes on the same antigen. Likewise, a tri-specific antibody stands for an antibody that can bind to three different types of antigen or three different epitopes on the same antigen.

“DBC” means-Dynamic binding capacity and is the binding capacity under operating conditions, i.e., in a packed affinity chromatography column during sample application. The DBC of a chromatography resin is the amount of target protein that binds to the resin under given flow conditions before a significant breakthrough of unbound protein occurs. DBC is determined by loading a sample containing a known concentration of the target protein and monitoring the flow-through. The protein will bind to the resin to a certain break point before unbound protein will flow through the column.

The DBC can be determined on the breakthrough curve at a loss of, for example, 10% protein. This is referred to as the Qb10% value, or simply Qb10%. A sample is applied to a chromatography resin column during a specific residence time and the dynamic binding capacity for each resin is calculated at 10% of the protein breakthrough i.e., the amount of target sample that is loaded onto the column until the concentration of target sample in the column effluent is 10% of the target sample concentration in the liquid sample. If the dynamic binding capacity for each resin is calculated at 80% of the breakthrough capacity, this is referred to as the Qb80% value

The term “liquid sample” as used herein, refers to a liquid containing at least one target substance which is sought to be purified from other substances also present. Liquid samples can, for example, be aqueous solutions, organic solvent systems, or aqueous/organic solvent mixtures or solutions. The source liquids are often complex mixtures or solutions containing many biological molecules (such as proteins, antibodies, hormones, and viruses), small molecules (such as salts, sugars, lipids, etc.) and even particulate matter. While a typical source liquid of biological origin may begin as an aqueous solution or suspension, it may also contain organic solvents used in earlier separation steps such as solvent precipitations, extractions, and the like. Examples of liquid samples that may contain valuable biological substances amenable to the purification by various embodiments of the present invention include, but are not limited to, a culture supernatant from a bioreactor, a homogenized cell suspension, plasma, plasma fractions, and milk. Alternatively, the liquid sample may be referred to as “Clarified Cell Culture Feed” or “CCF”.

A “buffer” is a substance which, by its presence in solution, increases the amount of acid or alkali that must be added to cause unit change in pH. A buffered solution resists changes in pH by the action of its acid-base conjugate components. Buffered solutions for use with biological reagents are generally capable of maintaining a constant concentration of hydrogen ions such that the pH of the solution is within a physiological range. The term “physiological pH” refers to the pH of mammalian blood (i.e., 7.38 or about 7.4). Thus, a physiologic pH range is from about 7.2 to 7.6. Traditional buffer components include, but are not limited to, organic and inorganic salts, acids and bases. Exemplary buffers for use in purification of biological molecules (e.g., protein molecules) include the zwitterionic or “Good” Buffers, see e.g., Good et al. (1966) Biochemistry 5:467 and Good and Izawa (1972) Methods Enzymol. 24:62.

The “equilibration buffer” is a buffer used to prepare the binding reagent, solid phase, or both, for loading of the source liquid containing the target protein. The equilibration buffer is preferably isotonic and commonly has a pH in the range from about 6 to about 8. The “loading buffer” is a buffer used to load the source liquid, or liquid sample, containing the binding region containing protein and impurities onto the solid phase to which the binding agent is immobilized. Often, the equilibration and loading buffers are the same.

“Washing liquid” or “wash buffer” as used herein all refer herein to the liquid used to carry away impurities from the chromatography resin to which is bound the target substance. More than one wash liquid can be employed sequentially, e.g., with the successive wash liquids having varying properties such as pH, conductivity, solvent concentration, etc., designed to dissociate and remove varying types of impurities that are non-specifically associated with the chromatography resin.

“Elution liquid” or “elution buffer”, which are used interchangeably herein, refers herein to the liquid that is used to dissociate the target substance from the chromatography resin, thereby eluting the binding region-containing protein from the immobilized binding agent, after it has been washed with one or more wash liquids. The elution liquid acts to dissociate the target substance without denaturing it irreversibly. Typical elution liquids are well known in the chromatography art and may have a different pH (typically lower pH), higher concentrations of salts, free affinity ligands or analogues, or other substances that promote dissociation of the target substance from the chromatography resin.

“Elution conditions” refers to process conditions imposed on the target substance-bound chromatography resin that dissociate the target substance from the chromatography resin, such as the contacting of the target substance-bound chromatography resin with an elution liquid or elution buffer to produce such dissociation.

Preferably the elution buffer has a low pH and thereby disrupts interactions between the kappa light chain binding separation matrix and the protein of interest. Preferably, the low pH elution buffer has a pH in the range from about 2 to about 5, most preferably in the range from about 3 to about 4. Examples of buffers that will control the pH within this range include glycine, phosphate, acetate, and citrate buffers, as well as combinations of these. The preferred such buffers are citrate and acetate buffers, most preferably sodium citrate or sodium acetate buffers.

Cleaning liquid may be an acidic solution or an alkali solution for removing resin residues after elution of the target substance. For instance precipitated proteins, hydrophobic proteins, nucleic acids, endotoxins and viruses may be removed by the cleaning liquid. Most commonly, alkali solutions are used for the purpose

Cleaning-in-place (CIP) is an important process for efficient use of a chromatography column. In order to maximize the number of cycles that a column can be reused, a cleaning procedure that efficiently removes impurities without being harmful to the chromatography resin is required.

As used herein, the terms “comprises”, “comprising”, “containing”, “having” and the like can mean “includes”, “including”, and the like; “consisting essentially of” or “consists essentially” is an open-ended term, allowing for the presence of more than that which is recited so long as basic or novel characteristics of that which is recited is not changed by the presence of more than that which is recited, but excludes prior art embodiments.

### DETAILED DESCRIPTION

The inventors had as an objective to invent a separation matrix containing Protein L-derived ligands having an improved stability towards alkaline cleaning procedures, while maintaining a satisfactory efficiency in binding capacity and flow characteristics in a chromatography setting, and preferably better than current commercially available separation matrices.

According to one aspect, the objective has been attained by providing a separation matrix comprising kappa light chain-binding ligands covalently coupled to a porous support, wherein said kappa light chain-binding ligands comprise, consists essentially of, or consists of multimers of alkali-stabilized Finegoldia (former Peptostreptococcus) Protein L domains; and said porous support is a convection-based chromatography matrix.

The kappa light chain-binding ligands comprised in the separation matrix of the present invention comprises, consists essentially of, or consists of, multimers of alkali-stabilized Finegoldia Protein L domains. The Protein L domains may be any functional Protein L derived domain as long as it is alkali stabilized. The Protein L domains are chosen from a functional variant of a B1 domain, a B2 domain, a B3 domain, a B4 domain, a B5 domain, a C2 domain, a C3 domain, a C4 domain and a D1 domain, wherein the positions which in an alignment corresponds to positions 10 and 45 in a B2 domain (SEQ ID NO 1) are histidine, and the position which in an alignment corresponds to position 60 in a B2 wt domain (SEQ ID NO 1) is a tyrosine or a glutamine. Thus, the above-mentioned positions corresponding to positions 10, 45 and 60 in a B2 wt domain (SEQ ID NO 1) are not variable within the functional Protein L domain.

(wt B2)
SEQ ID NO: 1
PKEEVTIKANLIYADGKTQTAEFKGTFEEAAAEAYRYADALKKDNGEYTVDVADKGYTLNIKFAGKEKTPEE
Examples of such functional Protein L domains may be:
(B2: N10H, N45H, N60Y mutations)
SEQ ID NO: 2
PKEEVTIKAHLIYADGKTQTAEFKGTFEEAAAEAYRYADALKKDHGEYTVDVADKGYTLYIKFAGKEKTPEE
(B2: N10H, N45H, N60Q mutations)
SEQ ID NO: 3
PKEEVTIKAHLIYADGKTQTAEFKGTFEEAAAEAYRYADALKKDHGEYTVDVADKGYTLQIKFAGKEKTPEE
(B3: N10H, N45H, N60Y mutations)
SEQ ID NO: 4
PKEEVTIKAH LIYADGKTQT AEFKGTFEEA TAEAYRYADL LAKEHGKYTV DVADKGYTLY IKFAGKEKTP EE
(B3: N10H, N45H, N60Q mutations)
SEQ ID NO: 5
PKEEVTIKAH LIYADGKTQT AEFKGTFEEA TAEAYRYADL LAKEHGKYTV DVADKGYTLQ IKFAGKEKTP EE
(B1: N10H, N45H, N60Y mutations)
SEQ ID NO: 6
SEEEVTIKAHLIFANGSTQTAEFKGTFEKATSEAYAYADTLKKDHGEYTVDVADKGYTLYIKFAGKEKTPEE
(B1: N10H, N45H, N60Q mutations)
SEQ ID NO: 7
SEEEVTIKAHLIFANGSTQTAEFKGTFEKATSEAYAYADTLKKDHGEYTVDVADKGYTLQIKFAGKEKTPEE
(B4: N10H, N45H, N60Y mutations)
SEQ ID NO: 8
PKEEVTIKAHLIYADGKTQTAEFKGTFAEATAEAYRYADLLAKEHGKYTADLEDGGYTIYIRFAGKKVDEKPEE
(B4: N10H, N45H, N60Q mutations)
SEQ ID NO: 9
PKEEVTIKAHLIYADGKTQTAEFKGTFAEATAEAYRYADLLAKEHGKYTADLEDGGYTIQIRFAGKKVDEKPEE
(B5: N9H, N44H, N59Y mutations)
SEQ ID NO 10
KEQVTIKEH IYFEDGTVQTATFKGTFAEATAEAYRYADLLSKEHGKYTADLEDGGYTIQIRFAGKEEPEE
(B5: N9H, N44H, N59Q mutations)
SEQ ID NO 11
KEQVTIKEHIYFEDGTVQTATFKGTFAEATAEAYRYADLLSKEHGKYTADLEDGGYTIQIRFAGKEEPEE
(C2b N57Y: N10H, N45H, N60Y mutations)
SEQ ID NO 12
PKEEVTIKVHLIFADGKTQTAEFKGTFEEATAKAYAYADLLAKEHGEYTADLEDGGYTIYIKFAGKETPETPEE
(C2b N57Y: N10H, N45H, N60Q mutations)
SEQ ID NO 13
PKEEVTIKVHLIFADGKTQTAEFKGTFEEATAKAYAYADLLAKEHGEYTADLEDGGYTIQIKFAGKETPETPEE
(C3b N39D, N57Y: N10H, N45H, N60Y mutations)
SEQ ID NO 14
PKEEVTIKVHLIFADGKIQTAEFKGTFEEATAKAYAYADLLAKEHGEYTADLEDGGYTIYIKFAGKETPETPEE
(C3b N39D, N57Y: N10H, N45H, N60Q mutations)
SEQ ID NO 15
PKEEVTIKVHLIFADGKIQTAEFKGTFEEATAKAYAYADLLAKEHGEYTADLEDGGYTIQIKFAGKETPETPEE
(C4: N10H, N45H, N60Y mutations)
SEQ ID NO: 16
PKEEVTIKVHLIFADGKTQTAEFKGTFEEATAEAYRYADLLAKVHGEYTADLEDGGYTIYIKFAGKEQPGENPG
(C4: N10H, N45H, N60Q mutations)
SEQ ID NO: 17
PKEEVTIKVHLIFADGKTQTAEFKGTFEEATAEAYRYADLLAKVHGEYTADLEDGGYTIQIKFAGKEQPGENPG
(D1: N10H, N45H, N60Y mutations)
SEQ ID NO: 18
PKEEVTIKAHLIFADGKTQTAEFKGTFEEATAEAYRYADLLAKVHGEYTADLEDGGYTIYIKFAGKEQPGEN
(D1: N10H, N45H, N60Q mutations)
SEQ ID NO: 19
PKEEVTIKAHLIFADGKTQTAEFKGTFEEATAEAYRYADLLAKVHGEYTADLEDGGYTIQIKFAGKEQPGEN

Preferably, the Protein L domain is selected from the group comprising of the B3 domain, the C2 domain, the C3 domain and the D-domain, wherein the positions which in an alignment corresponds to positions 10 and 45 in a B2 wt domain (SEQ ID NO 1) are histidine, and the position which in an alignment corresponds to position 60 in a B2 wt domain (SEQ ID NO 1) is a tyrosine or a glutamine. The above-mentioned positions corresponding to positions 10, 45 and 60 in a B2 wt domain (SEQ ID NO 1) are not variable within the functional Protein L domain.

The remaining positions in such a functional Protein L domain may be varied as long as the three-dimensional structure is not altered as compared to that of the B2 wt domain (SEQ ID NO 1), and as long as it at least retains the kappa light chain-binding capacity and is alkali-stabilized as compared to the B2 wt domain (SEQ ID NO 1). The variation may be conservative amino acid substitutions for an amino acid with a similar or identical charge, hydrophobicity, etc., and the skilled person is able to determine what such a variation of an amino acid may be.

The Protein L domain may have at least 90%, 95% or 98% sequence identity, or a 77.5% sequence similarity as determined by BLOSUM matrix of 75, with a gap open penalty of 12, a gap extension penalty of 3, with any one of the amino acid sequences SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18 or SEQ ID NO 19.

The functional Protein L domain may be a truncated sequence. For instance the positions corresponding to positions 1-4 in B2 wt domain (SEQ ID NO 1) may be deleted. For instance positions corresponding to positions following position 65 in B2 wt domain (SEQ ID NO 1) may be deleted.

(B1_trunc, N6H, N41H, N56Y mutations)
SEQ ID NO: 20
VTIKAHLIFANGSTQTAEFKGTFEKATSEAYAYADTLKKDHGEYTVDVADKGYTLYIKFAG
(B1_trunc, N6H, N41H, N56Q mutations)
SEQ ID NO: 21
VTIKAHLIFANGSTQTAEFKGTFEKATSEAYAYADTLKKDHGEYTVDVADKGYTLQIKFAG
(B2_trunc, N6H, N41H, N56Y mutations)
SEQ ID NO: 22
VTIKAHLIYADGKTQTAEFKGTFEEAAAEAYRYADALKKDHGEYTVDVADKGYTLYIKFAG
(B2_trunc, N6H, N41H, N56Q mutations)
SEQ ID NO: 23
VTIKAHLIYADGKTQTAEFKGTFEEAAAEAYRYADALKKDHGEYTVDVADKGYTLQIKFAG
(B3_trunc, N6H, N41H, N56Y mutations)
SEQ ID NO: 24
VTIKAHLIYADGKTQTAEFKGTFEEATAEAYRYADLLAKEHGKYTVDVADKGYTLYIKFAG
(B3_trunc, N6H, N41H, N56Q mutations)
SEQ ID NO: 25
VTIKAHLIYADGKTQTAEFKGTFEEATAEAYRYADLLAKEHGKYTVDVADKGYTLQIKFAG
(B4_trunc, N6H, N41H, N56Y mutations)
SEQ ID NO: 26
VTIKAHLIYADGKTQTAEFKGTFAEATAEAYRYADLLAKEHGKYTADLEDGGYTIYIRFAG
(B4_trunc, N6H, N41H, N56Q mutations)
SEQ ID NO: 27
VTIKAHLIYADGKTQTAEFKGTFAEATAEAYRYADLLAKEHGKYTADLEDGGYTIQIRFAG
(B5_trunc, N6H, N41H, N56Y mutations)
SEQ ID NO: 28
VTIKEHIYFEDGTVQTATFKGTFAEATAEAYRYADLLSKEHGKYTADLEDGGYTIYIRFAG
(B5_trunc, N6H, N41H, N56Q mutations)
SEQ ID NO: 29
VTIKEHIYFEDGTVQTATFKGTFAEATAEAYRYADLLSKEHGKYTADLEDGGYTIQIRFAG
(C2b N57Y_trunc, N6H, N41H, N56Y mutations)
SEQ ID NO: 30
VTIKVHLIFADGKTQTAEFKGTFEEATAKAYAYADLLAKEHGEYTADLEDGGYTIYIKFAG
(C2b N57Y_trunc, N6H, N41H, N56Q mutations)
SEQ ID NO: 31
VTIKVHLIFADGKTQTAEFKGTFEEATAKAYAYADLLAKEHGEYTADLEDGGYTIQIKFAG
(C3b N39D, N57Y trunc, N6H, N41H, N56Y mutations)
SEQ ID NO: 32
VTIKVHLIFADGKIQTAEFKGTFEEATAKAYAYADLLAKEHGEYTADLEDGGYTIYIKFAG
(C3b N39D, N57Y_trunc, N6H, N41H, N56Q mutations)
SEQ ID NO: 33
VTIKVHLIFADGKIQTAEFKGTFEEATAKAYAYADLLAKEHGEYTADLEDGGYTIQIKFAG
(C4_trunc, N6H, N41H, N56Y mutations)
SEQ ID NO: 34
VTIKVHLIFADGKTQTAEFKGTFEEATAEAYRYADLLAKVHGEYTADLEDGGYTIYIKFAG
(C4_trunc, N6H, N41H, N56Q mutations)
SEQ ID NO: 35
VTIKVHLIFADGKTQTAEFKGTFEEATAEAYRYADLLAKVHGEYTADLEDGGYTIQIKFAG
(D1_trunc, N6H, N41H, N56Y mutations)
SEQ ID NO: 36
VTIKAHLIFADGKTQTAEFKGTFEEATAEAYRYADLLAKVHGEYTADLEDGGYTIYIKFAG
(D1_trunc, N6H, N41H, N56Q mutations)
SEQ ID NO: 37
VTIKAHLIFADGKTQTAEFKGTFEEATAEAYRYADLLAKVHGEYTADLEDGGYTIQIKFAG

The Protein L domain may have at least 90%, 95% or 98% sequence identity, or a 77.5% sequence similarity as determined by BLOSUM matrix of 75, with a gap open penalty of 12, a gap extension penalty of 3, with any one of the amino acid sequences SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36 or SEQ ID NO:37.

As can be seen above, the C2-domain and the C3 domain comprise additional mutation(s). The C2 domain scaffold comprises an additional N57Y mutation and is herein named C2b. The C3 domain scaffold comprises additional N39D and N57Y mutations and is herein named C3b.

As specified above, the kappa light chain-binding ligands comprised in the separation matrix comprise, consists essentially of, or consists of multimers of the alkali-stabilized Protein L domains. The multimer may comprise two, three, four, five, six, seven, eight or nine alkali-stabilized Protein L domains. In an alternative language, the multimers may be a dimer, trimer, tetramer, pentamer, hexamer, heptamer, octamer or a nonamer. Preferably, the ligands comprise four, five, six or seven alkali-stabilized Protein L domains, such as five or six alkali-stabilized Protein L domains.

The multimer may further comprise a linker, spacer, or additional amino acid(s). The additional amino acid(s) may for instance originate from the cloning process and expression of the ligand or constitute a residue from a cleaved off signalling sequence. The skilled person will appreciate and understand that such additional amino acid(s) may vary without impacting the function of the kappa light chain-binding function of the multimer.

The porous support comprises a convection-based chromatography matrix. Said convection-based chromatography matrix may be a fibrous substrate. Said fibrous substrate may be based on electrospun polymeric fibres or cellulose fibres, optionally non-woven fibres, which in use form a stationary phase comprising a plurality of pores through which a mobile phase can permeate. The fibrous substrate may thus be a fibrous non-woven polymer matrix. Such a fibrous substrate can be found in a HiTrap Fibro™ unit from Cytiva™.

Convection-based chromatography support materials enable the combination of high flow rates with high binding capacity and are hence of interest for use in for example mAb purification.

The polymer fibers may be non-woven fibers. Using a randomly deposited fiber mat (non-woven) structure can encourage impeded flow thereby discouraging channeling.

The polymer fibers may be electrospun. Electrospinning provides fibers with consistent dimensions and can easily be tuned (for example, by varying atmospheric properties while spinning) to make fibers of different proportions. Electrospinning is a technique that also demonstrates excellent distribution properties, especially useful when creating layered membranes. Fiber mass transfer characteristics have been shown to be similar to those in a monolith structure which allow for flow rate independent separations

The polymer used in the present invention is not limited to any specific polymer and can be tailored for specific use. The polymer may be for example: nylon, poly(acrylic acid), polyacrylonitrile, polystyrene, polysulphone, polyacrylonitrile, polycaprolactone, collagen, chitosan, agarose and polyethylene oxide and combinations thereof. The polymer may be derivatised to enhance the solubility and/or other properties of the polymer in order to improve its suitability to be electrospun. The derivatised polymer (for instance polyether sulfone, cellulose acetate or poly(acrylonitrile-co-acrylic acid) can be treated post electrospinning to regenerate the original polymer or derivatise further to create a new functionality. The polymer used in the invention is typically cellulose. Cellulose is often used as it is readily available, cheap, biodegradable, biologically compatible and has a hydrophilic surface resulting in low non-specific binding.

The polymer fibres may be covalently cross-linked. Once an electrospun network of fibres has been made, the fibres may be fused together at points where fibres intersect one another by thermal, chemical or other methods. This leads to improved manual manipulation characteristics.

The fibres may have a diameter of 10 nm to 1000 nm. The fibres may have a diameter of 200 nm to 800 nm and may even have a diameter of 300 nm to 400 nm. Fibres of this size yield improved consistency of pore size and size distribution.

The fibres may have a mean length of greater then 10 cm. Fibres generated by electrospinning are typically much longer than the fibres found in conventional chromatography media. Longer fibres deliver improved layering properties. In some cases, where the electrospinning comprises a fibre emanating from a single source, a single continuous fibre may be produced and the membrane formed from this fibre alone, or from a small number (1, 2, 3, 4, 5, 6, 7, 8, 9, 10) of long fibres.

The pores of the stationary phase may be 10 nm to 10 μm in diameter, often 25 nm to 5 μm and can be 50 nm to 2 am in diameter. Use of pore sizes within these size ranges can help to minimise fouling of the chromatography medium and decrease product loss due to polarization, concentration and rejection at particle interfaces. However, the pores remain small enough to minimise the loss of target components passing through the membrane without coming into contact with the medium. The selection of these pore sizes ensures good utilisation of capacity and sharper breakthrough curves. It has previously been demonstrated that for a membrane structure with similar pore size the fibre structures were 1.5-2 times more permeable to aqueous flow than a traditional membrane produced by phase inversion. This is due to the relatively high surface porosity that the electrospinning process yields. It has previously been shown that a non-woven fibre membrane with average fibre diameter of 300 nm contained an average pore size of ca. 500 nm, yet yielding a porosity of 49%. By electrospinning fibre membranes, it is possible to achieve a high level of surface porosity of high distribution well above what could be expected from traditionally formed membranes as in their case the relationship of decreasing surface porosity with decreasing pore size is dominant.

The pores may have a narrow size distribution, wherein the standard deviation in pore diameter is preferably less than or equal to 250 nm. Pore size uniformity is one of several factors as well as, axial and radial diffusion and sorption kinetics, that has been shown to have an impact on key performance factors in chromatography (particularly affinity chromatography) such as breakthrough curve (BTC) sharpness.

The fibers as disclosed above are functionalized before the ligand is immobilized thereto. The preparation of a convective based chromatography matrix is disclosed in for instance WO/2019/137869 and WO/2013/068741, which are incorporated by reference in their entirety.

In the present disclosure “membrane” is often used interchangeably with the porous support. It should be clear that whenever “membrane” is used, it refers to the porous support material as disclosed above. The ligand density achieved on the porous support is at least 20 mg ligand/ml porous support, or at least 25 mg ligand/ml porous support, such as 30 mg ligand/ml porous support, such as at least 35 mg ligand/ml porous support, or at least 40 mg ligand/ml porous support, or at least 45 mg ligand/ml porous support, or at least 50 mg/ml porous support.

The separation matrix of the present has a dynamic binding capacity (DBC) of a kappa light chain-comprising antibody, such as Trastuzumab, of 25 g/mL at 10% breakthrough when running at a flow of 10 mL/min in a 0.4 mL HiTrap™ device. According to one preferred embodiment, the separation matrix of the present has a dynamic binding capacity (DBC) of a kapa light chain-comprising antibody, such as Trastuzumab, of 45 g/mL at 10% breakthrough when running at a flow of 10 mL/min in a 0.4 mL HiTrap™ device.

The present invention also relates to methods for isolation of a kappa light chain-binding protein. Said method is preferably performed with the separation matrix as disclosed above. The isolation may be from a cell culture or other liquid comprising kappa light-chain binding proteins.

Thus, the present disclosure provides for a method of isolating a kappa light chain-containing protein comprising the steps of:
a) contacting a liquid sample comprising a kappa light chain-containing protein with a separation matrix;
b) washing said separation matrix with one or a combination of several washing liquids;
c) eluting the kappa light chain-containing protein from the separation matrix with an elution liquid; and
d) cleaning the separation matrix with a cleaning liquid.

The separation matrix has a dynamic binding capacity (DBC) of the kappa light chain-containing protein, such as Trastuzumab, of 28 g/mL at 10% breakthrough when running at a flow of 10 mL/min in a 0.4 mL HiTrap™ device. According to one embodiment the separation matrix as disclosed above is used in said method.

The generation of bispecific antibodies or IgG molecules is difficult as the pairing of the light and heavy chains, and consequently the variable domains therein (VL; VH) may be promiscuous. The pairing of two different light and two different heavy chains may lead to a large number of mispairing, as normally only one specific asymmetric combination is wanted, and a multitude of combinations achieved will be non-functional or unwanted molecules, such as for instance monospecific homodimers. Thus, there is an increasing need for improved tools and methods to separate bispecific antibodies from monospecific antibodies, as well as separating mismatched bispecific antibodies from correctly matched bispecific antibodies. This may be done by separation based on different light chains comprised in a bispecific antibody.

According to yet another aspect, there is provided herein a method for separation of bispecific antibodies, comprising the steps of:
a) contacting a liquid sample comprising kappa light chain-containing proteins with a separation matrix,
b) washing said separation matrix with one or a combination of several washing liquids,
c) eluting the kappa light chain-containing protein from the separation matrix with an elution liquid and
d) cleaning the separation matrix with a cleaning liquid.

The separation matrix has a dynamic binding capacity (DBC) of the kappa light chain-containing protein, such as Trastuzumab, of 28 g/mL at 10% breakthrough when running at a flow of 10 mL/min in a 0.4 mL HiTrap™ device.

According to one embodiment the separation matrix as disclosed above is used in said method.

The elution step may be performed with a volume gradient. This is shown in Example X. Alternatively, the elution step may be performed with a pH gradient.

This method enables to separate bi-specific antibodies from monospecific antibodies, or mismatched antibodies, based on the presence of a kappa light chain. A monoclonal antibody has two identical kappa light chain. A bispecific antibody can be designed to comprise two different kappa light chain.

Any antibody not comprising a kappa light chain, such as two lambda light chains, will not bind the separation matrix and consequently be present in the effluent flow during step a) or be washed away during step b). Any antibodies that have at least one kappa light chain will bind to the separation matrix. Upon elution, antibodies that have only one kappa light chain will elute prior to antibodies that have two kappa light chains.

By using the separation matrix as disclosed above, which binds to subclass 1, 3 or 4 kappa light chains, it is also possible to separate antibodies or antibody fragments comprising one or two kappa light chains of subclass 2 from antibodies or antibody fragments comprising one or two kappa light chains of subclass 1, 3 or 4. By the same principle as above, an antibody comprising two kappa light chains of subclass 2, or one kappa light chain of subclass 2 and a lambda light chain, will not bind the separation matrix and consequently be present in the effluent flow during step a) or be washed away during step b). An antibody with one kappa light chain of subclass 2 and one kappa light chain of any of subclasses 1, 3 or 4 will elute prior to an antibody with two kappa light chains of any of subclasses 1, 3 and 4.

### EXAMPLES

The general method for preparing the material used in the examples below is:
1: Obtaining a substrate formed by one or more polymer fibers (Cellulose Acetate (CA))
2. Subjecting the CA to Glycidol polymerization and saponification
3: Functionalization of the material from step 2 with Divinyl sulfone (DVS)
4: Immobilization of kappa light chain-binding ligands on to the material obtained in step 3 comprising;
   Reduction and desalting of kappa light chain-binding ligands;
   Immobilization of reduced kappa light chain-binding ligands on to the material obtained from step 3 on a shaking table;
   Washing of the kappa light chain-binding ligand-immobilized material
5: Deactivation of residual vinyl-groups using 1-Thioglycerol
6: Washing the material to remove as much as possible of the non-bound kappa light chain-binding ligands.

The steps were performed, unless otherwise specified herein, in accordance with the methods disclosed in Example 6 in WO/2019/137869 and WO/2018/011600.

### Protein Reduction

To a protein solution, sodium sulfate (1.4 M) was added and when all had dissolved, dithiothreitol (DTT, 0.1 M) was added. The reaction mixture was put in a shaking table (23° C., 500 rpm) and left to reduce overnight

### Desalting

The desalting was performed by using Pre-packed PD10 columns containing Sephadex-G-25 medium. The columns were equilibrated with 4 column volumes of de-gassed desalting solution (0.15 NaCl, 1 mM EDTA) prior to loading the protein (max 2.5 mL). The eluted fractions were collected and combined.

The desalted solution was diluted 20 times by desalting solution and the absorbance at 276 nm was measured and corrected by a blank of desalting buffer. By using the determined protein concentration, it was then possible to calculate the desired amount of desalted protein solution to be used during immobilization.

For immobilization, solutions with a concentration of 25-30 mg ligand/ml was used and added to the material activated according to the methods referred to above. Depending on temperature, pH and reaction time for the immobilization of the ligands, from 20 mg ligand up to at least 50 mg ligand could be immobilized per ml of porous support material. A longer reaction time as in Example 6 in WO/2019/137869 and WO/2018/011600 will generally lead to higher ligand density than a shorter reaction time as in example 5 in the same disclosures.

### Example 1—Dynamic Binding Capacity

A separation matrix according to the present invention was prepared as above, and the Dynamic binding capacity was analysed.

### Sample Preparation

500 mL or 250 mL IgG at 0.5 mg/mL in 20 mM Tris-HCl, 150 mM NaCl, pH 7.5 (binding buffer) was prepared by diluting 1.515 mL or 0.758 mL of Gammanorm (165 mg/mL) in binding buffer and adjusting the volume to 500 mL or 250 mL in a volumetric flask. Just prior to the analysis, the Gammanorm solution was filtrated using a Sterivex filter. After filtration was the concentration checked by measuring UV absorbance at 280 nm. Calculation of IgG concentration was done according to Beer's law:

$$A = ε L c$$

where A is the UV absorption at 280 nm, E is the absorptivity coefficient (for polyclonal IgG ε is 1.38 ml/(mg×cm). L is the path length of the cell holder. c is the concentration of the solution.

3.4 mL of purified Trastuzumab (30 g/L) was diluted with 20 mM Phosphate 150 mM NaCl pH 7.2 in a total volume of 200 mL. The concentration was determined by UV measurements at 280 nm using 96 well UV plate, 200 μL/well. Blank was 20 mM Phosphate 150 mM NaCl pH 7.2. The Trastuzumab concentration was calculated using the extinction coefficient 1.48.

dAB is an domain antibody comprising the variable light chain and was produced according to the method described in the article “Recombinant production of a V_{L} single domain antibody in Escherichia coli and analysis of its interaction with Peptostreptococcal protein L” (Protein Expression and Purification, Volume 51, Issue 2, February 2007, Pages 253-259).

3.45 mL of dAb (14.5 g/L) was diluted with 20 mM Phosphate 150 mM NaCl pH 7.2 in a total volume of 100 mL. The concentration was determined by UV measurements at 280 nm using 96 well UV plate, 200 μL/well. Blank was 20 mM Phosphate 150 mM NaCl pH 7.2. The dAb concentration was calculated using the extinction coefficient 1.6.

The ligands were coupled to the porous support as disclosed in Example 6 in WO/2019/137869 and WO/2013/068741.

TABLE 1
Buffers

| Buffer | Composition |
|---|---|
| Equilibration/binding | 20 mM Phosphate 150 mM NaCl pH 7.2 |
| Wash of unbound protein | 20 mM Phosphate 500 mM NaCl pH 7 |
| Wash of salt | 50 mM Citrate pH 5 |
| Elution | 50 mM Citrate pH 2.3 |
| CIP | 0.1M NaOH |
| Re-equilibration | 20 mM Phosphate 150 mM NaCl pH 7.2 |
| pH adjustment of eluates | 500 mM Phosphate pH 8 |
| to approximately pH 7 | |

A membrane single disc of the porous support coupled with ligand was installed in a device and connected to a chromatographic system, ÄKTAavant 25 IP31154, 10 mm cell. The membrane was equilibrated with the equilibration/binding buffer before protein sample load with gammanorm or Trastuzumab, respectively. The capacity at 10% breakthrough (Qb10%) was calculate and reported. The flow used during equilibration and washing of the membrane was 20 mL/minutes. The flow was reduced during protein load to 10 mL/min. The analysis was performed on duplicate membrane discs and each disc was analyzed two times. The first run was a blank without protein load followed by two frontal analysis runs with protein.

The breakthrough capacity was calculated using Extensions-DBC Calculations-Analyze, which is an Evaluation tool in Unicorn software used with the ÄKTAavant 25 IP31154.

For calculation of breakthrough capacity at 10% (Qb10), equation below was used. That is i.e. the amount of Trastuzumab that is loaded onto the column until the concentration of Trastuzumab in the column effluent is 10% of the Trastuzumab concentration in the liquid sample.

$${q}_{10 \%} = \frac{{C}_{0}}{{V}_{C}} [ {V}_{app} - {V}_{sys} - {\int }_{{V}_{sys}}^{{V}_{app}} \frac{A ( V ) - {A}_{sub}}{{A}_{100 \%} - {A}_{sub}} ⋆ dv ]$$

$${A}_{100 \%} = 100 \% UV signal$$

$${A}_{sub} = absorbance contribution from non - binding mAb$$

$$A ( V ) = absorbance at a given applied volume$$

$${V}_{C} = column volume$$

$${V}_{app} = volume applied until 10 \% breakthrough$$

$${V}_{sys} = system dead volume$$

$${C}_{0} = liquid sample concentration$$

For all the experiments, the sample solution comprised 0.5 mg protein.

TABLE 2
Dynamic Binding Capacity Trastuzumab results of two prototypes

| | Qb10% | Qb10% | Delta p | Delta p |
|---|---|---|---|---|
| | mg/mL | mg/mL | pressure MPa | pressure MPa |
| Run | Test 1 | Test 2 | Test 1 | Test 2 |
| 1 | 17.33 | 21.97 | 0.32 | 0.39 |
| 2 | 12.31 | 17.83 | 0.33 | 0.4 |
| 3 | 15.92 | 21.77 | 0.32 | 0.4 |
| 4 | 10.8 | 16.84 | 0.33 | 0.41 |
| Average | 14.09 | 19.60 | 0.33 | 0.40 |

The DBC decreases between run 1 and 2 and increases again for run 3 then drops for run 4 again. This is due to the fact that no blank run was done between the analyses 1 and 2 and between 3 and 4, respectively.

TABLE 3
Dynamic Binding Capacity Trastuzumab Results

| | Qb10% | Qb10% | Delta p | Delta p |
|---|---|---|---|---|
| | mg/mL | mg/mL | pressure MPa | pressure MPa |
| Run | Test 1 | Test 2 | Test 1 | Test 2 |
| 1 | 15.38 | 21.4 | 0.32 | 0.41 |
| 2 | 15.49 | 21.44 | 0.32 | 0.41 |
| Average | 15.44 | 21.42 | 0.32 | 0.41 |

In this experiment a blank run was added between the two runs 1 and 2. This shows that a blank run should be performed between each run.

Without being bound to any theory, it seems that the higher DBC achieved in test 2 may be attributed to the fact that a CIP with 0.1 M NaOH has been performed on the material before the test 2, whereas test 1 is performed without a preceding CIP wash.

TABLE 4
Dynamic Binding Capacity dAb Results of prototype

| | | | Average | | Delta p | Average |
|---|---|---|---|---|---|---|
| | Qb10% | Qb10% | of | Delta p | pressure | of Delta p |
| | mg/ml | mg/ml | Qb10% | pressure | MPa Test | pressure |
| Run | run Test 1 | run Test 2 | mg/ml | MPa Test 1 | 2 | MPa |
| 1 | 12.77 | 12.99 | 12.88 | 0.33 | 0.33 | 0.33 |
| 2 | 9.41 | 10.54 | 9.98 | 0.44 | 0.54 | 0.49 |

TABLE 5
Dynamic Binding Capacity dAb Results of
Re-run After CIP 0.1M NaOH with prototype

| | Qb10% | Qb10% | Delta p | Delta p |
|---|---|---|---|---|
| | mg/mL | mg/mL | pressure MPa | pressure MPa |
| Run | Test 1 | Test 2 | Testt 1 | Test 2 |
| 1 | 10.53 | 13.79 | 0.34 | 0.55 |
| 2 | 9.85 | 13.31 | 0.34 | 0.55 |
| Average | 10.19 | 13.55 | 0.34 | 0.55 |

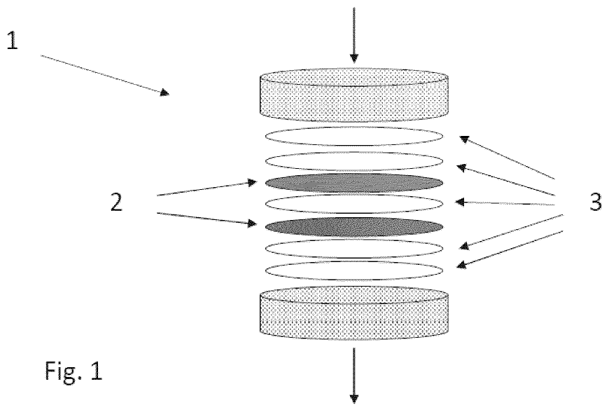

Next, the DBC of prototypes according to the present invention were compared to a commercial product with the same convection-based matrix. The matrix was comprised in a HiTrap™ Device as shown in FIG. 1. The HiTrap™ prototype devices (1) have a volume of 0.4 mL. The device (1) contains two stacked Fibro prototype membranes (2) with non-woven material layers (3) in between as filling material. The disc diameter is 26.4 mm. Prototype 1 comprised a separation matrix with 52 mg ligand/ml membrane. Prototype 2 comprised a separation matrix with 34 mg ligand/ml membrane.

TABLE 6
DBC comparison with commercial product and protypes in a HiTrap™ device.

| | DBC (Qb 5) g/mL | | | DBC (Qb 10) g/mL | | | | | |
|---|---|---|---|---|---|---|---|---|---|
| | | | | | | | deltaC (Mpa) | | |
| | run | run | Average | run | run | Average | run | run | Average |
| Test | 1 | 2 | Qb5 | 1 | 2 | Qb10 | 1 | 2 | delta C |
| Fibro™ | 27,6 | 27,2 | 27,4 | 28,6 | 28,3 | 28,4 | 0,16 | 0,16 | 0,16 |
| PrismA | | | | | | | | | |
| (Cytiva™) | | | | | | | | | |
| Prototype 1 | 48,3 | 47,5 | 47,9 | 49,9 | 49,1 | 49,5 | 0,28 | 0,28 | 0,28 |
| Prototype 2 | 27,1 | 27,7 | 27,4 | 28,2 | 28,8 | 28,5 | 0,19 | 0,19 | 0,19 |

As can be seen above, the prototypes exhibit at least as good a DBC as the commercial product Fibro™ PrismA, both for Qb5 and Qb10.

### Example 2—Resolution Between Fab-Fragment and Trastuzumab Based on Volume Gradient

It was further investigated if resolution between Fab-fragment and Trastuzumab could be achieved on a prototype according to the present invention.

The Fab fragment (Fab) was produced from Trastuzumab by papain cleavage. The Trastuzumab solution was adjusted to pH 7.4 by addition of 0.5 M Sodium phosphate and then diluted 1+1 in digestion buffer (25 mM Na-phosphate, 1 mM EDTA, 5 mM mercapto-ethanol, pH 7.5). Final volume was approx. 100 mL Papain crystals were added to the solution. The solution was incubated at 37° C. over-night. Thereafter, Antipain (papain inhibitor) was added to the digested Trastuzumab. The solution was left in room temperature for 30 min prior to application onto a Capto™ L HiScale 26 column to remove Fc-containing molecules (Fc or partially digested Trastuzumab, collected in flow through). Fab was collected during elution.

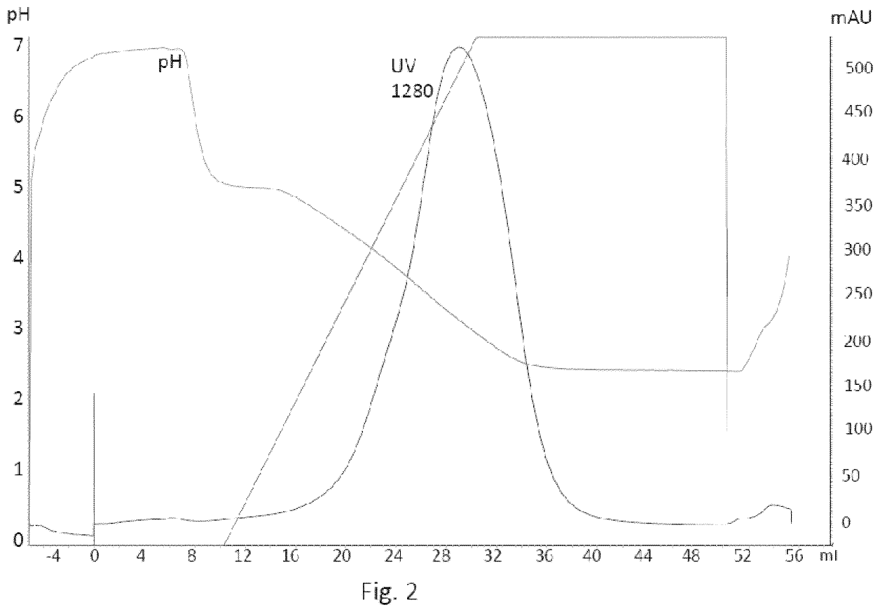

A linear pH gradient from pH 5.0 to 2.3 with 50 mM Citrate buffer was used to separate Fab and Trastuzumab. Resolution experiments were done from 20 mL to 100 mL linear gradient, e.g. the gradient was increased from 20 mL to 40, 60 and 100 mL, with a run time from 5.5 to 13.5 min. The results are shown in FIG. 2-5.

The results show that Fab and Trastuzumab co-elutes when the pH gradient slope was 20 mL, FIG. 2.

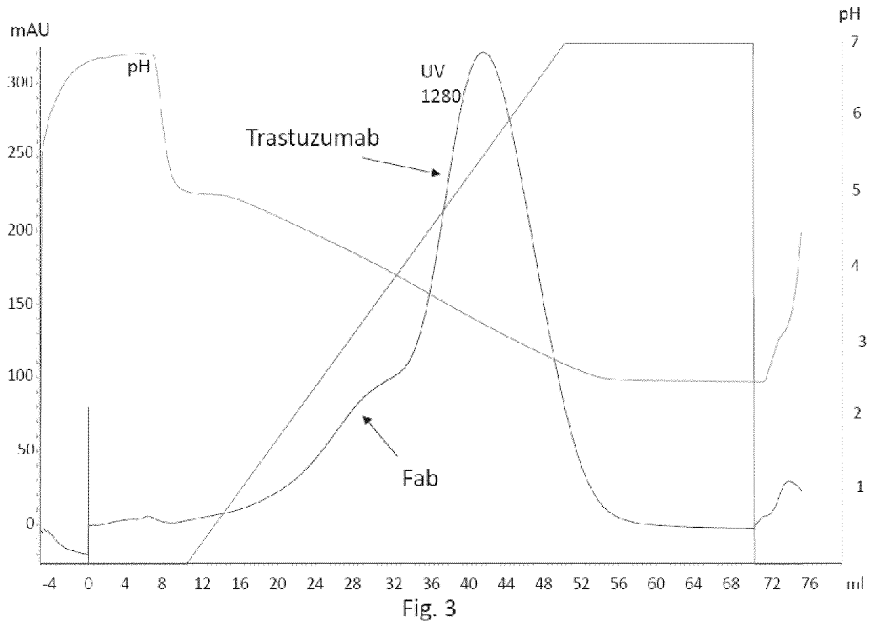

The shoulder on the peak increases when the gradient is flattened and increased to 40 mL for the separation between Fab and Trastuzumab on the prototype, FIG. 3.

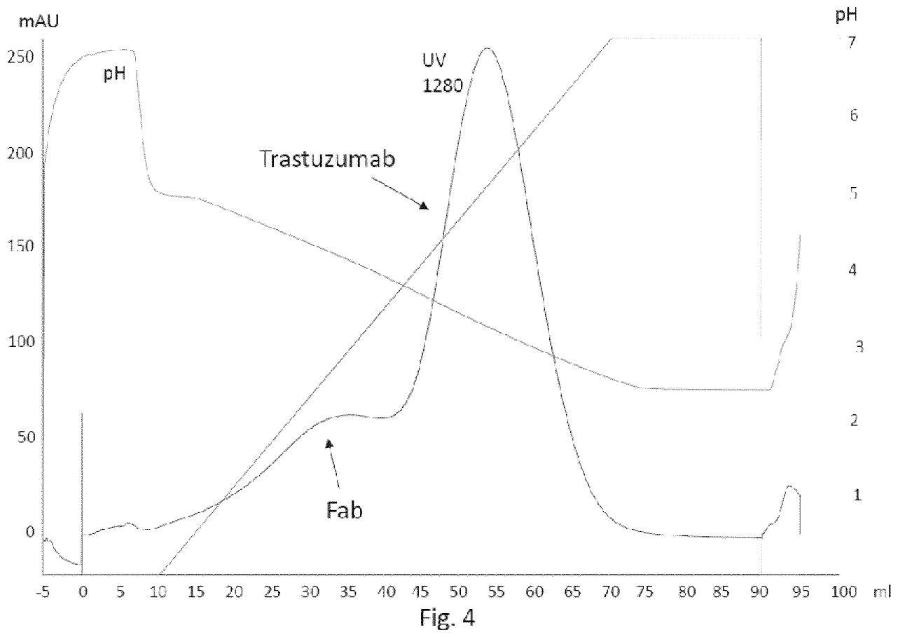

A better separation between Fab and Trastuzumab is obtained when pH gradient slope is 60 mL, FIG. 4.

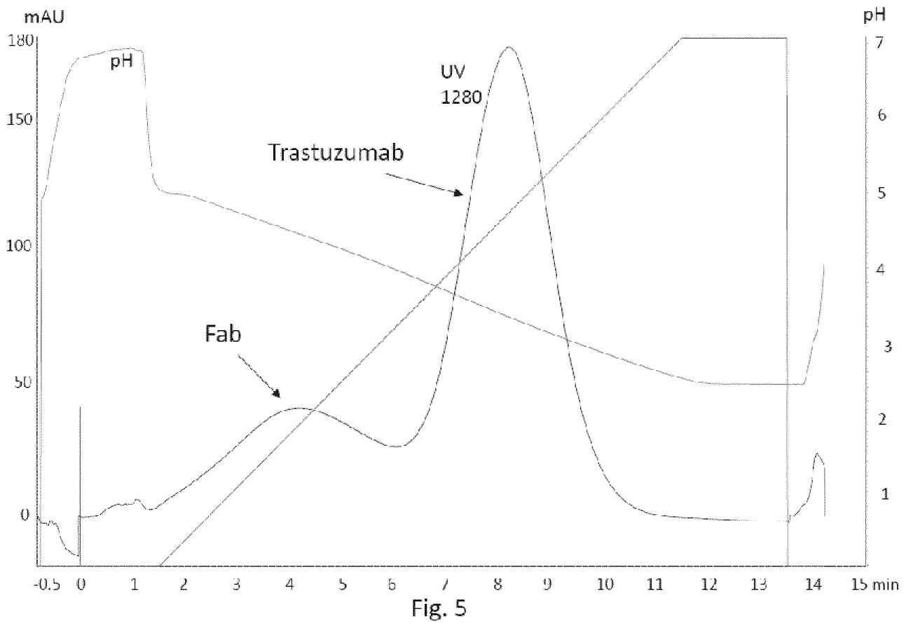

And an even better separation, with a resolution of 0.88, between Fab and Trastuzumab is achieved at pH gradient slope 100 mL on Fibro. See FIG. 5, where the Fab peak is located in the left and the Trastuzumab peak in the right of the chromatogram.

1. A separation matrix comprising kappa light chain-binding ligands covalently coupled to a porous support, wherein
said kappa light chain-binding ligands comprise, consists essentially of, or consists of multimers of alkali-stabilized Finegoldia magna (formerly Peptostreptococcus Magnus) Protein L domains; and
said porous support is a convection-based chromatography matrix.

2. The separation matrix according to claim 1, wherein the convection-based chromatography matrix is a fibrous substrate.

3. The separation matrix according to claim 2, wherein the fibrous substrate is based on electrospun polymeric fibers or cellulose fibers, optionally non-woven fibers.

4. The separation matrix according to claim 3, wherein the polymer is selected from the group consisting of cellulose, cellulose acetate, polysulfones, polyamides, polyacrylic acid, polymethacrylic acid, polyacrylonitrile, polystyrene, polyethylene oxide, and mixtures thereof.

5. The separation matrix according to claim 3, wherein the fibrous substrate is a fibrous non-woven polymer matrix.

6. The separation matrix according to claim 2, wherein the fibers comprised in said fibrous substrate have a cross-sectional diameter of 10-1000 nm, such as 200-800 nm, 200-400 nm or 300-400 nm.

7. The separation matrix according to claim 2, wherein the ligands are bound to divinyl sulfone functional groups coupled to glycidol groups grafted onto the fibrous substrate.

8. The separation matrix according to claim 1, wherein said kappa light chain-binding ligands comprise at least two alkali-stabilized Protein L domains.

9. The separation matrix according to claim 8, wherein the alkali-stabilized Protein L domains are selected from the group comprising of functional variants of a B1 domain, a B2 domain, a B3 domain, a B4 domain, a B5 domain, a C2 domain, a C3 domain, a C4 domain and a D1 domain of Finegoldia magna (formerly Peptostreptococcus Magnus) Protein L, wherein the positions which in an alignment corresponds to positions 10 and 45 in a B2 domain (SEQ ID NO 1) are histidine, and the position which in an alignment corresponds to position 60 in a B2 domain (SEQ ID NO 1) is a tyrosine or a glutamine.

10. The separation matrix according to claim 9, wherein the alkali-stabilized Protein L domains are chosen from the group comprising a B2 domain, a B3 domain, a B4 domain, a C2 domain, a C3 domain, a C4 domain and a D1 domain.

11. The separation matrix according to claim 8, wherein the alkali-stabilized Protein L domains have at least 90%, 95% or 98% sequence identity or a 77.5% sequence similarity as determined by BLOSUM matrix of 75, with a gap open penalty of 12, a gap extension penalty of 3, with any one of the amino acid sequences SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18 or SEQ ID NO 19, wherein the positions which in an alignment corresponds to positions 10 and 45 in SEQ ID NO 1, and the position which in an alignment corresponds to position 60 in SEQ ID NO 1 are not variable.

12. The separation matrix according to claim 8, wherein the alkali-stabilized Protein L domains have at least 90%, 95% or 98% sequence identity, or a 77.5% sequence similarity as determined by BLOSUM matrix of 75, with a gap open penalty of 12, a gap extension penalty of 3, with any one of the amino acid sequences SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36 or SEQ ID NO:37.

13. The separation matrix according to claim 1, wherein the ligand density is at least 20 mg/ml porous support, or at least 25 mg/ml porous support, or at least 30 mg/ml porous support, or at least 35 mg/ml porous support, or at least 40 mg/ml porous support, or at least 45 mg/ml porous support, or at least 50 mg/ml porous support.

14. The separation matrix according to claim 1, having a dynamic binding capacity (DBC) of a kappa light chain-comprising antibody, such as Trastuzumab, of 25 g/mL at 10% breakthrough when running at a flow of 10 mL/min in a 0.4 mL HiTrap™ device.

15. The separation matrix according to claim 1, having a dynamic binding capacity (DBC) of a kappa light chain-comprising antibody, such as Trastuzumab, of 45 g/mL at 10% breakthrough when running at a flow of 10 mL/min in a 0.4 mL HiTrap™ device.

16. A method of isolating a kappa light chain-containing protein comprising the steps of:
a) contacting a liquid sample comprising a kappa light chain-containing protein with a separation matrix;
b) washing said separation matrix with one or a combination of several washing liquids;
c) eluting the kappa light chain-containing protein from the separation matrix with an elution liquid; and
d) cleaning the separation matrix with a cleaning liquid;
wherein the separation matrix has a dynamic binding capacity (DBC) of a kappa light chain-comprising antibody, such as Trastuzumab, of 25 g/mL at 10% breakthrough when running at a flow of 10 mL/min in a 0.4 mL HiTrap™ device.

17. A method for separation of isolating a kappa light chain-binding protein from lambda light chain-containing proteins, comprising the steps of
a) contacting a liquid sample comprising a kappa light chain-containing protein with a separation matrix;
b) washing said separation matrix with one or a combination of several washing liquids;
c) eluting the kappa light chain-containing protein from the separation matrix with an elution liquid; and
d) cleaning the separation matrix with a cleaning liquid;
wherein the separation matrix has a dynamic binding capacity (DBC) of a kappa light chain-comprising antibody, such as Trastuzumab, of 25 g/mL at 10% breakthrough when running at a flow of 10 mL/min in a 0.4 mL HiTrap™ device.

18. A method for separation of bispecific antibodies from mono-specific antibodies, comprising the steps of:
a) contacting a liquid sample comprising a bispecific antibody with a separation matrix;
b) washing said separation matrix with one or a combination of several washing liquids;
c) eluting the bispecific antibody from the separation matrix with an elution liquid; and
d) cleaning the separation matrix with a cleaning liquid;
wherein the separation matrix has a dynamic binding capacity (DBC) of a kappa light chain-comprising antibody, such as Trastuzumab, of 25 g/mL at 10% breakthrough when running at a flow of 10 mL/min in a 0.4 mL HiTrap™ device.

19. The method of claim 17, wherein the separation is performed by applying a volume gradient or a pH gradient in step c).

20. (canceled)
